(19) United States
(12) Patent Application Publication
(10) Pub. No.: US 2024/0407401 A1
(43) Pub. Date: Dec. 12, 2024

(54) METHOD FOR REGULATING A TRANSPORT VELOCITY IN A PASTEURIZER AND A DEVICE FOR CARRYING OUT THE METHOD

(71) Applicant: Krones AG, Neutraubling (DE)

(72) Inventors: Stefan KAATZ, Fleckeby (DE); Jan MUENZER, Harrislee (DE); Jesse SCHMUCK, Flensburg (DE)

(21) Appl. No.: 18/736,333

(22) Filed: Jun. 6, 2024

(30) Foreign Application Priority Data
Jun. 6, 2023 (DE) 10 2023 114 812.5

(51) Int. Cl. A23L 3/00; A23L 3/005

(52) U.S. Cl. CPC A23L 3/003; A23L 3/005

(57) ABSTRACT

A method for regulating a transport velocity in a pasteurizer comprises regulating of the transport velocity by changing, such as reducing or increasing, the transport velocity, making a corresponding first change, such as reducing or increasing, of a first number of pasteurization zones by a second number, and making a corresponding second change, such as increasing or reducing, of a third number of cooling zones by the second number. A device for carrying out the method comprises: a pasteurizer having a first number of pasteurization zones and a third number of cooling zones, a transport device for transporting containers at a transport velocity, a control device configured for the changing of the transport velocity, making the corresponding first change, and making the corresponding second change.

### CROSS REFERENCE TO RELATED APPLICATION

The present application claims priority to German Patent Application No. 10 2023 114 812.5 filed on Jun. 6, 2023. The entire contents of the above-listed application are hereby incorporated by reference for all purposes.

### TECHNICAL FIELD

The disclosure relates to a method for regulating a transport velocity in a pasteurizer and a device for carrying out the method.

### BACKGROUND

EP 2 833 742 B1 discloses a pasteurizer having a pasteurizer-internal transport element for moving containers filled with filling material through multiple zones situated in succession in the transport direction, in which zones the containers are subjected to a liquid treatment medium having different treatment temperatures. The treatment temperatures rise in stages in a group of zones that form preheating zones in the transport direction from zone to zone. In at least one subsequent pasteurization zone, the treatment temperature corresponds to the pasteurization temperature and in subsequent cooling zones decreases from zone to zone in a stepped manner in the direction of transport. The transport velocity of the transport element is continuously controllable or regulatable. When the transport velocity increases, the number of zones serving as preheating zones is reduced while the treatment temperature in these zones is simultaneously increased, and the number of zones serving as pasteurization zones is increased. When the transport velocity is reduced, the number of zones serving as preheating zones is increased with partial reduction of the treatment temperatures, and the number of zones acting as pasteurization zones is reduced.

### SUMMARY

### Object

The object of the disclosure is to provide a method for regulating a transport velocity in a pasteurizer and a device for carrying out the method, which method can enable achieving a desired pasteurization.

### Achievement

The object is achieved by the method for regulating a transport velocity in a pasteurizer and the device for carrying out the method as described herein.

The method according to the disclosure for regulating a transport velocity in a pasteurizer comprises a regulation of the transport velocity by changing, such as reducing or increasing, the transport velocity, a corresponding first change, such as reducing or increasing a first number of pasteurization zones of the pasteurizer by a second number, and a corresponding second change, such as increasing or reducing a third number of cooling zones of the pasteurizer by the second number.

The change in the transport velocity may be continuous. For example, it may be provided that no stepwise changing of the transport velocity takes place.

The transport velocity may be changed starting from each transport velocity (wherein each transport velocity can be seen here in the possible region of the pasteurizer), for example continuously. For example, it may be provided that no stepwise changing of the transport velocity takes place.

The cooling zones may be arranged in a transport direction of containers through the pasteurizer downstream of the pasteurization zones. The containers may be transported through the pasteurizer by means of a transport device and may thus pass through the pasteurization and cooling zones. In the pasteurization and cooling zones, treatment medium with different temperatures may be sprayed onto the containers by means of respective spray devices.

For example, when the transport velocity is reduced, the last pasteurization zone in the transport direction may become a cooling zone (for example by a corresponding adaptation of the temperature of the treatment medium). The pasteurization zones located in the transport direction upstream of the last pasteurization zone, which may become the cooling zone, may continue to be operated as pasteurization zones. For example, the pasteurization temperature may be increased in them.

The temperature of at least the last pasteurization zone, which can become the cooling zone, may be adjusted when the transport velocity is reduced. It can thus be achieved that product present in the containers can be sufficiently pasteurized despite the reduced transport velocity, i.e., for example, can receive a predefined number of pasteurization units. An underpasteurization or overpasteurization can be avoided. In addition, a pasteurization of the product at low temperatures may be avoided by converting the last pasteurization zone into a cooling zone and maintaining pasteurization units upstream in the transport direction.

The reduction of the transport velocity may preclude a stoppage of the pasteurizer, i.e., a reduction to a velocity of zero. The reduction of the transport velocity may preclude a reverse movement, i.e., a transport of containers counter to the transport direction.

Because the regulation of the transport velocity may be performed, for example continuously, by changing the number of pasteurization zones and cooling zones during operation, no following stops are required in order to optionally be able to achieve a desired number of pasteurization units.

For example, no prediction is required as to whether a reduction or an increase in the transport velocity possibly takes place in order to be able to carry out a regulation. The method, as described here further above or below, may be carried out without a (for example, time) prediction for a regulation or a change of the transport velocity.

The method may further comprise maintaining a fourth number of heating zones of the pasteurizer. The heating zones may be arranged upstream of the pasteurization zones in a transport direction of containers through the pasteurizer. For example, the heating zones may have a stepwise increasing treatment temperature of a treatment medium in the transport direction of the containers. The treatment medium may be water having the corresponding treatment temperature. When the transport velocity is reduced, an increase in the temperatures of the treatment medium in the individual heating zones may be provided, wherein the stepwise temperature increase from heating zone to heating zone can be maintained. When the transport velocity is increased, a reduction in the temperatures of the treatment medium in the individual heating zones may be provided, wherein the stepwise temperature rise from heating zone to heating zone can be maintained.

Reducing the first number of pasteurization zones of the pasteurizer by the second number and increasing the third number of cooling zones of the pasteurizer by the second number may comprise a continuous lowering of a first temperature in the last pasteurization zone, whereby the last pasteurization zone may become a first cooling zone.

The continuous lowering of a first temperature in the last pasteurization zone may comprise converting a supply of a spray device in the last pasteurization zone with treatment medium from the last pasteurization zone into a supply of the spray device with treatment medium from the first cooling zone, and converting a supply of a spray device in the last heating zone with treatment medium from the first cooling zone into a supply of the spray device with treatment medium from the last pasteurization zone.

By integrating the last pasteurization zone as a spray cascade with the first cooling zone (that cooling zone which can be arranged downstream of the last pasteurization zone in the transport direction) into a recuperation of the heating and cooling zones, the desired temperature decrease in the last pasteurization zone can take place. The heat removed in this way from the last pasteurization zone may be supplied to the last heating zone.

The more the transport velocity is reduced, the more pasteurization zones can gradually be integrated into the spray cascade and become cooling zones.

The method may further comprise an interruption of a separate supply of heated water into the last pasteurization zone. The heated treatment medium may be provided by a heat exchanger.

The reduction of the first number of pasteurization zones of the pasteurizer by the second number and the increase of the third number of cooling zones of the pasteurizer by the second number can be accomplished by means of regulating multiple regulatable valves. For example, the multiple regulatable valves may be arranged in a pressure piping system of spray devices. For example, the multiple regulatable valves may comprise flap valves.

By providing the valves in the pressure piping system of the corresponding spray devices, a sufficient pressure of the treatment medium and thus a uniform spray pattern of the spray devices can be ensured.

The transport velocity may be reduced continuously, for example infinitely variably, or the transport velocity may be reduced in 0.5%, 1% or 1.5% increments.

The change may comprise increasing the transport velocity, the corresponding first change, an increase in the first number of pasteurization zones of the pasteurizer by the second number, and the corresponding second change, a reduction of the third number of cooling zones of the pasteurizer by the second number.

Increasing the first number of pasteurization zones of the pasteurizer by the second number and increasing the third number of cooling zones of the pasteurizer by the second number may comprise continuously increasing a first temperature in the first cooling zone, whereby the first cooling zone may become a last pasteurization zone.

The method may comprise reducing the transport velocity and then increasing the transport velocity or vice versa. Between the reduction and increase or increase and reduction of the transport velocity, the transport velocity may have, for example, a constant value for a period of time. The time period does not need to be predetermined. It may result, for example, from the regulation.

A device for carrying out the method, as described above or further below, comprises a pasteurizer having a first number of pasteurization zones and a third number of cooling zones, a transport device which is designed to transport containers at a transport velocity through the pasteurizer in a transport direction, and a control device configured for changing, such as reducing or increasing, the transport velocity, a corresponding first change, such as reducing or increasing the first number of pasteurization zones of the pasteurizer by a second number, and a corresponding second change, such as increasing or reducing the third number of cooling zones of the pasteurizer by the second number.

The cooling zones may be arranged in a transport direction of containers through the pasteurizer downstream of the pasteurization zones. The containers may be transported through the pasteurizer by means of a transport device and may thus pass through the pasteurization and cooling zones. In the pasteurization and cooling zones, treatment medium with different temperatures may be sprayed onto the containers by means of respective spray devices.

The pasteurizer may comprise a fourth number of heating zones.

The heating zones may be arranged upstream of the pasteurization zones in a transport direction of containers through the pasteurizer. For example, the heating zones may have a stepwise increasing treatment temperature of a treatment medium in the transport direction of the containers. The treatment medium may be water having the corresponding treatment temperature. When the transport velocity is reduced, an increase in the temperatures of the treatment medium in the individual heating zones may be provided, wherein the stepwise temperature increase from heating zone to heating zone can be maintained. When the transport velocity is increased, a reduction in the temperatures of the treatment medium in the individual heating zones may be provided, wherein the stepwise temperature rise from heating zone to heating zone can be maintained.

The device may comprise multiple regulatable valves.

The reduction or increase of the first number of pasteurization zones of the pasteurizer by the second number and the increase or reduction of the third number of cooling zones of the pasteurizer by the second number can take place by means of regulating the multiple regulatable valves.

The multiple regulatable valves may be arranged in a pressure piping system of spray devices. For example, the multiple regulatable valves may comprise flap valves.

The device may further comprise a conversion device for converting a supply of a spray device in the last pasteurization zone with treatment medium from the last pasteurization zone into a supply of the spray device with treatment medium from the first cooling zone, and further a conversion device for converting a supply of a spray device in the last heating zone with treatment medium from the first cooling zone into a supply of the spray device with treatment medium from the last pasteurization zone. The conversion devices may comprise the multiple regulatable valves. The conversion device may be used, for example, when the transport velocity is reduced if the first number of pasteurization zones of the pasteurizer is to be reduced by the second number and the third number of cooling zones of the pasteurizer is to be increased by the second number.

The device may further comprise a heat exchanger for heating treatment medium. The heated treatment medium can be supplied to the last pasteurization zone.

The device may further comprise a further heat exchanger for cooling treatment medium.

### BRIEF DESCRIPTION OF THE FIGURES

The accompanying figures show, by way of example, aspects and/or exemplary embodiments of the disclosure for better understanding and illustration. In the figures:

FIG. 1 shows a diagram with temperature curves of a pasteurization and a curve of the recorded pasteurization units at 100% transport velocity,

FIG. 2 shows a diagram with temperature curves of a pasteurization and a curve of the recorded pasteurization units at 80% transport velocity,

FIG. 3 shows a diagram with temperature curves of a pasteurization and a curve of the recorded pasteurization units at 50% transport velocity,

FIG. 4 shows a switching arrangement of a spray cascade at 100% transport velocity,

FIG. 5 shows a switching arrangement of the spray cascade of the pasteurizer which was described in FIG. 4, wherein the second pasteurization zone is cooled,

FIG. 6 shows a switching arrangement of the spray cascade of the pasteurizer which was described in FIG. 4, wherein the second pasteurization zone is cooled by means of the first cooling zone,

FIG. 7 shows a switching arrangement for a displacement of a recuperation zone at 100% transport velocity,

FIG. 8 shows a switching arrangement for a displacement of the recuperation zone at 80% transport velocity, and

FIG. 9 shows a switching arrangement for a displacement of the recuperation zone at 50% transport velocity.

### DETAILED DESCRIPTION OF FIGURES

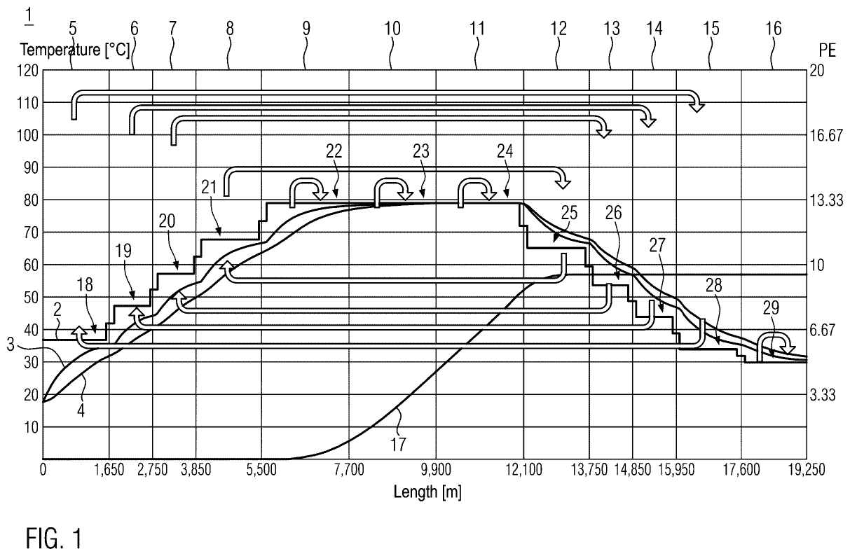

FIG. 1 shows a diagram 1 with temperature curves 2, 3, 4 of a pasteurization and a curve 17 of the recorded pasteurization units PE at 100% transport velocity. 100% transport velocity can be in this case a nominal transport velocity, i.e., the transport velocity can correspond to the transport velocity during normal operation of the pasteurizer.

The pasteurization may be carried out in a pasteurizer through which containers filled with product can be transported while arranged, for example, on a transport device, with the transport velocity for a pasteurization process in a transport direction. The described pasteurizer comprises a total of twelve zones 5-16 through which the containers pass. The corresponding temperature of the treatment medium prevailing in a zone results in a classification into heating zone, pasteurization zone or cooling zone.

At 100% transport velocity, the first four zones 5, 6, 7, 8 serve as heating zones 18, 19, 20, 21, the fifth, sixth and seventh zone 9, 10, 11 each serve as a pasteurization zones 22, 23, 24 and the eighth to twelfth zone 12, 13, 14, 15, 16 each serve as cooling zones 25, 26, 27, 28, 29.

In the pasteurizer, the four heating zones 18-21 are passed through first in succession and have treatment temperatures of a treatment medium that rise stepwise from the first zone 5 to the second zone 6 to the third zone 7 and to the fourth zone 8. The treatment medium may be water having the corresponding treatment temperature, which water can be sprayed onto the containers by means of spray devices arranged in the respective zones. In the first zone 5, a temperature of the treatment medium which can be sprayed onto the containers may be 36.6° C., in the second zone 6 it may be 47.2° C., in the third zone 7 it may be 57.2° C., and in the fourth zone 8 it may be 67.7° C.

After the four heating zones 18-21, the containers may pass through the three pasteurization zones 22-24 in which the treatment temperatures of the treatment medium in the fifth zone 9, sixth zone 11 and seventh zone 12 correspond to a pasteurization temperature, which may be 78.8° C., for example. The increase in the recorded pasteurization units PE which begins in the first pasteurization zone 22 and progresses continuously in the second pasteurization zone 23 and the third pasteurization zone 24 is apparent from the curve 17 of the recorded pasteurization units PE. The recorded pasteurization units are indicated in the unit PE over the length covered in the pasteurizer in meters.

After the pasteurization zones 22-24, the containers can pass through the five cooling zones 25-29, wherein the treatment temperatures of the treatment medium decrease from the eighth zone 12 to the ninth zone 13 to the tenth zone 14 to the eleventh zone 15 and to the twelfth zone 16 in a stepped manner. In the eighth zone 12, a temperature of the treatment medium which can be sprayed onto the containers may be 65.0° C., in the ninth zone 13 it may be 53.7° C., in the tenth zone 14 it may be 43.8° C., in the eleventh zone 15 it may be 33.9° C., and in the twelfth zone 16 it may be 29.9° C. Pasteurization units PE may continue to be recorded in the first cooling zone 25 by the temperatures prevailing in the container at the beginning; only after the end of the first cooling zone 25 does the number of recorded pasteurization units then remain constant. The pasteurization units PE achieved after pasteurization of a container may have a value of 9.5.

In the diagram, the curve profile of the treatment temperature for the spray devices is denoted by the reference sign 2. The treatment temperature is indicated in degrees Celsius over the length covered in the pasteurizer in meters.

In addition, the temperature profile of the product in the interior of the containers is measured at a point within one third of the height of the containers from the bottom, indicated in the curve with the reference sign 4. The product temperature is indicated in degrees Celsius over the length covered in the pasteurizer in meters.

Also, the curve profile of the mean temperature is represented by the reference sign 3. The average temperature is indicated in degrees Celsius over the length covered in the pasteurizer in meters.

For the distribution of the treatment medium to the spray devices of the respective zones of the pasteurizer, it is provided that treatment medium is supplied from the eighth zone 12 to the spray device of the fourth zone 8. A treatment medium from a zone is to be understood as meaning such a treatment medium as is collected in a basin or the like, for example after spraying onto containers below this zone, and from there can be supplied to a spray device via a corresponding pipeline system.

Treatment medium from the ninth zone 13 is supplied to the spray device of the third zone 7. Treatment medium from the tenth zone 14 is supplied to the spray device of the second zone 6. Treatment medium from the eleventh zone 15 is supplied to the spray device of the first zone 6. Treatment medium from the twelfth zone 16 is supplied to the spray device of the twelfth zone 16.

In the three pasteurization zones 22-24, the treatment medium is fed back from one of the zones to the respective zone. There is no exchange of treatment medium between these zones 22-24.

A recuperation of heat of the treatment medium may take place by the treatment medium being supplied from the first zone 5 to the spray device of the eleventh zone 15, by the treatment medium being supplied from the second zone 6 to the spray device of the tenth zone 14, by the treatment medium being supplied from the third zone 7 to the spray device of the ninth zone 13, and by the treatment medium being supplied from the fourth zone 8 to the spray device of the eighth zone 12.

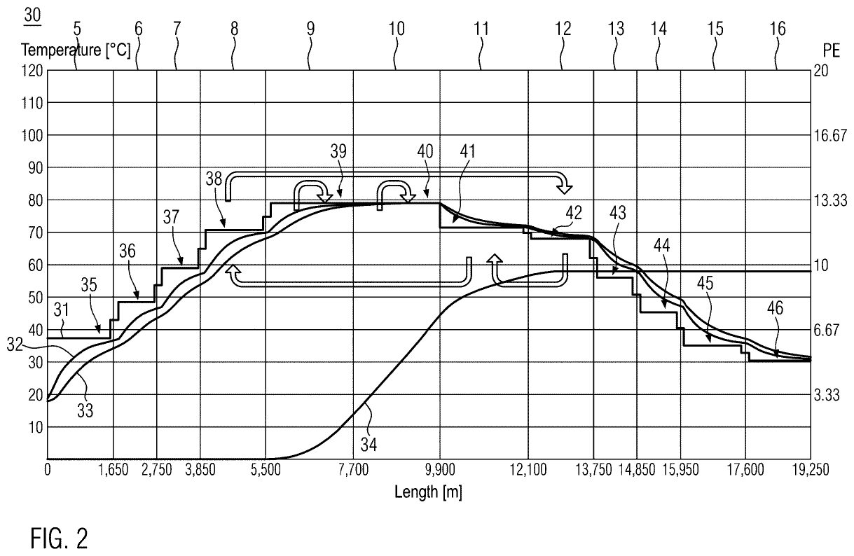

FIG. 2 shows a diagram 30 with temperature curves 31, 32, 33 of a pasteurization and a curve 34 of the recorded pasteurization units PE at 80% transport velocity. 80% transport velocity in this case can mean a transport velocity, which can correspond to 80% of the nominal transport velocity. 80% transport velocity can be required during operation of the pasteurizer if disturbances occur. Disturbances can occur if a machine upstream of the pasteurizer can transfer too few containers (compared to a number of containers during normal operation) to the pasteurizer and/or if a machine downstream of the pasteurizer can transport away too few containers (compared to a number of containers during normal operation).

When the transport velocity is reduced from 100% to 80%, the regulation of the transport velocity in the pasteurizer can be accomplished by reducing by one zone the number of pasteurization zones of the pasteurizer which are provided at 100% transport velocity and increasing the number of cooling zones of the pasteurizer by one zone accordingly. This can be done by switching of one or more valves, for example flap valves, which can be arranged in pipelines between the zones and the spray devices. At 80% transport velocity, just as at 100% transport velocity, the first four zones 5-7, 8 serve as heating zones 35, 36, 37, 38, the fifth and sixth zones 9, 10 each serve as pasteurization zones 39, 40 and the seventh to twelfth zones 12-16 each serve as cooling zones 41, 42, 43, 44, 45, 46.

The third pasteurization zone, which was described in FIG. 1, was thus converted into the first cooling zone, so that six cooling zones 41-46 are now present, but only two pasteurization zones 39, 40 are still present. The reduction in the number of pasteurization zones and the corresponding increase in the number of cooling zones can be effected by integrating the third pasteurization zone of FIG. 1 into a spray cascade by opening or closing corresponding valves. These valves may be provided in the pressure piping system of the corresponding spray device in order to ensure a sufficient pressure of the treatment medium and thus a uniform spray pattern of the spray devices.

First, just as at 100% transport velocity, the four heating zones 35-38 of containers are passed in succession. The number of heating zones 35-38 is not changed when the transport velocity is reduced. The treatment temperature of the treatment medium in the individual heating zones is used for the spray devices; however, at 80% transport velocity said treatment temperature is different from the temperature values at 100% transport velocity. In the first heating zone 35, a temperature of the treatment medium that can be sprayed onto the containers can be 37.5° C., in the second heating zone 36 it can be 48.5° C., in the third heating zone 37 it can be 59.0° C., and in the fourth heating zone 38 it can be 70.8° C.

Following the four heating zones 35-38 are the two pasteurization zones 39, 40 in which the treatment temperature of the treatment medium that is used for the spray device corresponds to a pasteurization temperature, which can be 79.0° C., for example, in the two pasteurization zones 39, 40. The increase in the recorded pasteurization units PE, which begins in the first pasteurization zone 39 and progresses continuously in the second pasteurization zone 40, can be seen on the curve 34 of the recorded pasteurization units PE. The recorded pasteurization units are indicated in the unit PE over the length covered in the pasteurizer in meters.

The six cooling zones 41-46 follow the two pasteurization zones 39, 40, wherein the treatment temperature of the treatment medium used for the spray device decreases in a stepped manner in the successive cooling zones 41-46. In the first cooling zone 41, a temperature of the treatment medium that can be sprayed onto the containers can be 71.5° C., in the second cooling zone 42 it can be 68.1° C., in the third cooling zone 43 it can be 56.1° C., in the fourth cooling zone 44 it can be 45.5° C., in the fifth cooling zone 45 it can be 35.2° C., and in the sixth cooling zone 46 it can be 30.7° C. In the first cooling zone 41 and the second cooling zone 42, pasteurization units PE can continue to be recorded by the temperatures prevailing in the container; only toward the end of the second cooling zone 42 does the number of recorded pasteurization units remain constant. The pasteurization units PE achieved after pasteurization of a container can have a value of 9.7.

In the diagram, the curve profile of the treatment temperature for the spray devices is denoted by the reference sign 31. The treatment temperature is indicated in degrees Celsius over the length covered in the pasteurizer in meters.

In addition, the temperature profile of the product in the interior of the containers is measured at a point within one third of the height of the containers from the bottom, indicated in the curve with the reference sign 33. The product temperature is indicated in degrees Celsius over the length covered in the pasteurizer in meters.

Also, the curve profile of the mean temperature is represented by the reference sign 32. The average temperature is indicated in degrees Celsius over the length covered in the pasteurizer in meters.

For the distribution of the treatment medium to the spray devices of the respective zones of the pasteurizer, it is provided that treatment medium is supplied from the eighth zone 12 to the spray device of the seventh zone 11, that treatment medium is supplied from the seventh zone 11 to the spray device of the fourth zone 8, that treatment medium is supplied from the ninth zone 13 to the spray device of the third zone 7, that treatment medium is supplied from the tenth zone 14 to the spray device of the second zone 6, that treatment medium is supplied from the eleventh zone 15 to the spray device of the first zone 5, and that the treatment medium is supplied from the twelfth zone 16 to the spray device of the twelfth zone 16.

In the two pasteurization zones 39, 40, the treatment medium is supplied from one of the zones to the respective zone. There is no exchange of treatment medium between these zones.

A recuperation of heat of the treatment medium may take place by the treatment medium being supplied from the first zone 5 to the spray device of the eleventh zone 15, by the treatment medium being supplied from the second zone 6 to the spray device of the tenth zone 14, by the treatment medium being supplied from the third zone 7 to the spray device of the ninth zone 13, and by the treatment medium being supplied from the fourth zone 8 to the spray device of the eighth zone 12.

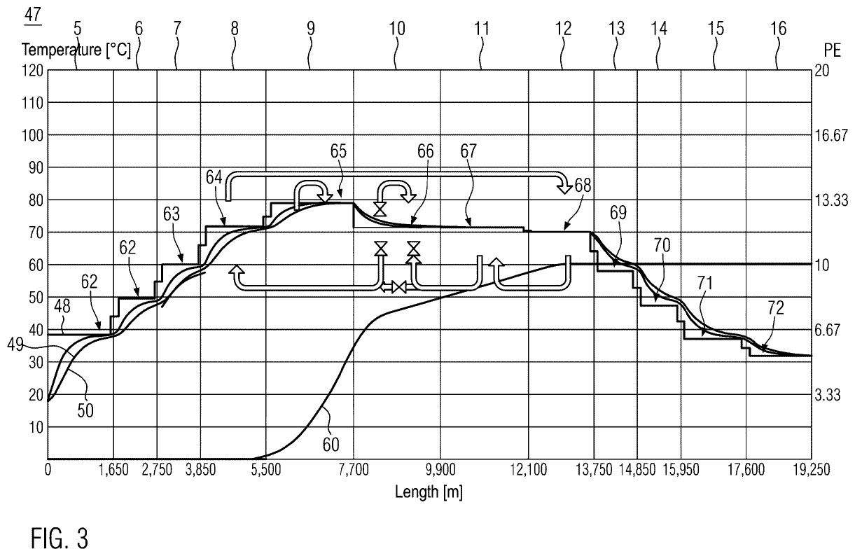

FIG. 3 shows a diagram 47 with temperature curves 48, 49, 50 of a pasteurization and a curve 34 of the recorded pasteurization units PE at 50% transport velocity. 50% transport velocity can in this case mean a transport velocity which may correspond to 50% of the nominal transport velocity. 50% transport velocity may be required during operation of the pasteurizer if disturbances, such as those described in reference to FIG. 2, occur to an increased extent, for example.

When the transport velocity is reduced from 80% to 50%, the regulation of the transport velocity in the pasteurizer may be accomplished by further reducing the number of pasteurization zones of the pasteurizer, which are provided at 80% transport velocity, by one zone, and by increasing the number of cooling zones of the pasteurizer by one zone accordingly.

The second pasteurization zone, which was described in FIG. 2, was thus converted into the first cooling zone, so that now there are seven cooling zones 66, 67, 68, 69, 70, 71, 72, but only one pasteurization zone 65. The reduction in the number of pasteurization zones and the corresponding increase in the number of cooling zones may be effected by integrating the second pasteurization zone of FIG. 2 into a spray cascade by opening or closing corresponding valves. These valves may be provided in the pressure piping system of the corresponding spray device in order to ensure a sufficient pressure of the treatment medium and thus a uniform spray pattern of the spray devices.

First, just as at 100% transport velocity, the containers successively pass through the four heating zones 61, 62, 63, 64. The number of heating zones is not changed when the transport velocity is reduced. However, the temperature values of the treatment temperatures of the treatment medium in the individual zones are different at 50% transport velocity than the temperature values at 80% transport velocity. In the first heating zone 61, a temperature of the treatment medium which may be sprayed onto the containers may be 38.6° C., in the second heating zone 62 it may be 49.4° C., in the third heating zone 63 it may be 60.1° C., and in the fourth heating zone 64 it may be 71.5° C.

Following the four heating zones 61-64 is a pasteurization zone 65, in which the treatment temperature of the treatment medium corresponds to a pasteurization temperature, which may be 78.8° C., for example. The increase in the recorded pasteurization units PE which begins in the pasteurization zone 65 may be seen on the curve 60 of the recorded pasteurization units PE. The recorded pasteurization units are indicated in the unit PE over the length covered in the pasteurizer in meters.

Following the one pasteurization zone 65 are seven cooling zones 66-72, wherein the treatment temperatures of the treatment medium that is used for the spraying device decrease in a step-like manner in the successive cooling zones 66-72. In the first cooling zone 66, a temperature of the treatment medium that can be sprayed onto the containers may be 71.3° C., in the second cooling zone 67 it may be 70.8° C., in the third cooling zone 68 it may be 70° C., in the fourth cooling zone 69 it may be 58.2° C., in the fifth cooling zone 70 it may be 47.5° C., in the sixth cooling zone 71 it may be 37.3° C., and in the seventh cooling zone 46 it may be 31.6° C. In the first cooling zone 66, the second cooling zone 67 and the third cooling zone 68, further pasteurization units PE may then be recorded by the temperatures prevailing in the container; only toward the end of the third cooling zone 68 does the number of recorded pasteurization units remain constant. The pasteurization units PE achieved after pasteurization of a container may have a value of 10.1.

In the diagram, the curve profile of the treatment temperature for the spray devices is marked with the reference sign 48. The treatment temperature is indicated in degrees Celsius over the length covered in the pasteurizer in meters.

In addition, the temperature profile of the product in the interior of the containers is measured at a point within one third of the height of the containers from the bottom, indicated in the curve with the reference sign 50. The product temperature is indicated in degrees Celsius over the length covered in the pasteurizer in meters.

Also, the curve profile of the mean temperature is represented by the reference sign 49. The average temperature is indicated in degrees Celsius over the length covered in the pasteurizer in meters.

For the distribution of the treatment medium to the spray devices of the respective zones of the pasteurizer, it is provided that treatment medium is supplied from the eighth zone 12 to the spray device of the seventh zone 11, that treatment medium is supplied from the seventh zone 11 to the spray device of the sixth zone 10 and/or of the fourth zone 8, that treatment medium is supplied from the sixth zone 10 to the spray device of the fourth zone 8, that treatment medium is supplied from the ninth zone 13 to the spray device of the third zone 7, that treatment medium is supplied from the tenth zone 14 to the spray device of the second zone 6, that treatment medium is supplied from the eleventh zone 15 to the spray device of the first zone 5, and that the treatment medium is supplied from the twelfth zone 16 to the spray device of the twelfth zone 16.

In the one pasteurization zone 65, the treatment medium is fed back from the pasteurization zone 65 to the spray device of this zone.

In the sixth zone 10, the treatment medium is supplied from the sixth zone 10 to the spray device of the sixth zone 10.

A recuperation of heat of the treatment medium may take place by the treatment medium being supplied from the first zone 5 to the spray device of the eleventh zone 15, by the treatment medium being supplied from the second zone 6 to the spray device of the tenth zone 14, by the treatment medium being supplied from the third zone 7 to the spray device of the ninth zone 13, and by the treatment medium being supplied from the fourth zone 8 to the spray device of the eighth zone 12.

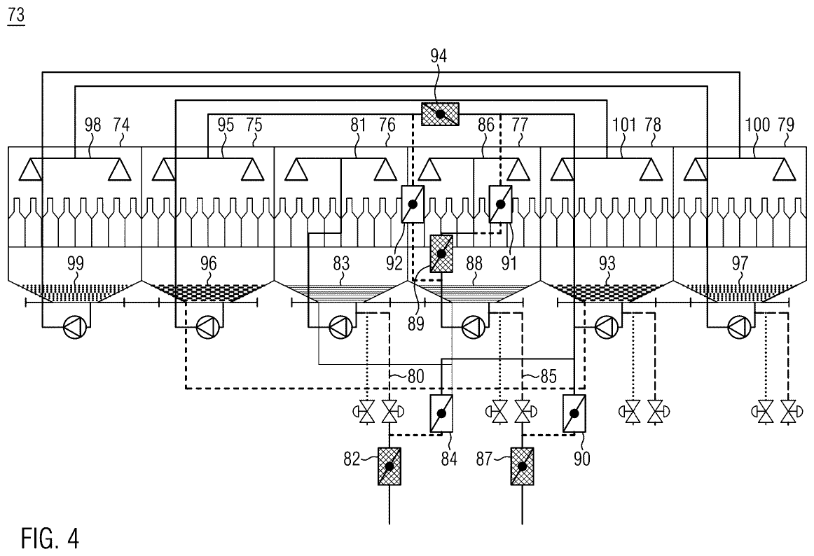

FIG. 4 shows a switching arrangement 73 of a spray cascade at 100% transport velocity in a pasteurizer, which comprises two heating zones 74, 75, two pasteurization zones 76, 77, and two cooling zones 78, 79. An operation with 100% transport velocity can be regarded as normal operation.

The supply line 80 to the spray device 81 of the first pasteurization zone 76 may be supplied with heated treatment medium via an open first valve 82, which medium is mixed with treatment medium 83 from the first pasteurization zone 76 and may then be supplied to the spray device 81 of the first pasteurization zone 76. A third valve 84 is closed. The heated treatment medium may be provided by a heat exchanger.

The supply line 85 to the spray device 86 of the second pasteurization zone 77 may be supplied with heated treatment medium via an open second valve 87, which medium can be mixed with treatment medium 88 from the second pasteurization zone 77, and may then be supplied to the spray device 86 of the second pasteurization zone 77 via an open fifth valve 89. A fourth valve 90, a sixth valve 91 and a seventh valve 92 are closed. The heated treatment medium may be provided by the heat exchanger or a further heat exchanger.

From the first cooling zone 78, treatment medium 93 is supplied to the spray device 95 of the second heating zone 75 via an open eighth valve 94.

From the second heating zone 75, treatment medium 96 is supplied to the spray device 101 of the first cooling zone 78.

From the second cooling zone 79, treatment medium 97 is supplied to the spray device 98 of the first heating zone 74.

From the first heating zone 74, treatment medium 99 is supplied to the spray device 100 of the second cooling zone 79.

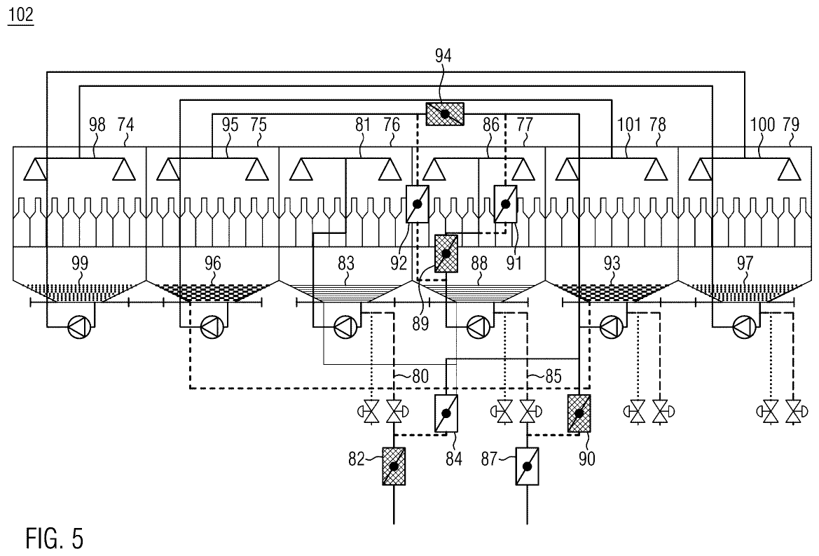

FIG. 5 shows a switching arrangement 102 of the spray cascade of the pasteurizer which was described in FIG. 4, wherein the second pasteurization zone 77 is cooled.

The supply line 80 to the spray device 81 of the first pasteurization zone 76 may be supplied with heated treatment medium via the open first valve 82, which medium is mixed with treatment medium 83 from the first pasteurization zone 76 and may then be supplied to the spray device 81 of the first pasteurization zone 76. The heated treatment medium may be provided by the heat exchanger.

The supply line 103 to the spray device 95 of the second heating zone 75 may be supplied with heated treatment medium via the open fourth valve 90, which medium can be mixed with treatment medium 93 from the first cooling zone 78 and may then be supplied to the spray device 95 of the second heating zone 75 via the open eighth valve 94. The heated treatment medium may be provided by the heat exchanger or the further heat exchanger.

Treatment medium 96 from the second heating zone 75 is supplied to the first cooling zone 78.

Treatment medium 88 from the second pasteurization zone 77 may be supplied to the spray device 86 of the second pasteurization zone 77 via the open fifth valve 89.

From the second cooling zone 79, treatment medium 97 is supplied to the spray device 98 of the first heating zone 74.

From the first heating zone 74, treatment medium 99 is supplied to the spray device 100 of the second cooling zone 79.

The second valve 87, third valve 84, sixth valve 91 and seventh valve 92 are closed.

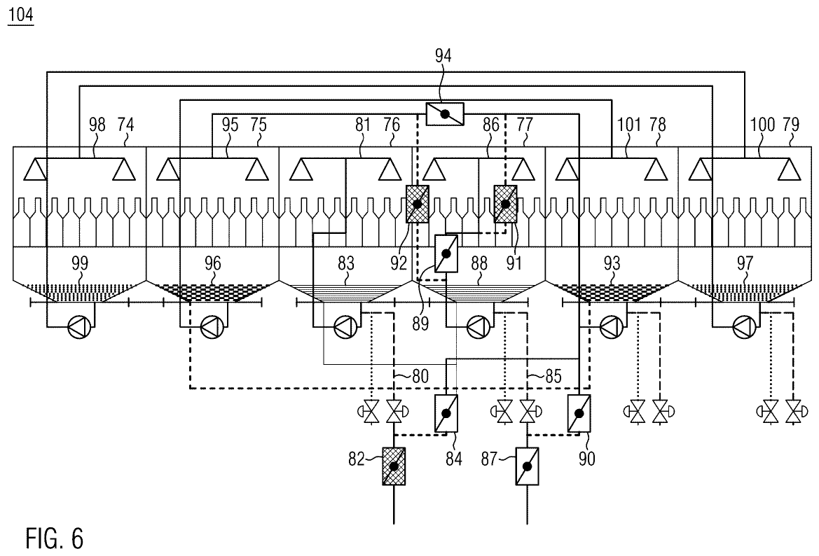

FIG. 6 shows a switching arrangement 104 of the pasteurizer which was described in FIG. 4, wherein the second pasteurization zone 77 is cooled by means of the first cooling zone 78.

The supply line 80 to the spray device 81 of the first pasteurization zone 76 may be supplied with heated treatment medium via the open first valve 82, which medium is mixed with treatment medium 83 from the first pasteurization zone 76 and may then be supplied to the spray device 81 of the first pasteurization zone 76. The heated treatment medium may be provided by the heat exchanger.

Treatment medium 88 may be supplied from the second pasteurization zone 77 to the spray device 95 of the second heating zone 75 via the open seventh valve 92.

Treatment medium 96 may be supplied from the second heating zone 75 to the spray device 101 of the first cooling zone 78.

Treatment medium 93 may be supplied from the first cooling zone 78 to the spray device 86 of the second pasteurization zone 77 via the open sixth valve 91.

From the second cooling zone 79, treatment medium 97 is supplied to the spray device 98 of the first heating zone 74.

From the first heating zone 74, treatment medium 99 is supplied to the spray device 100 of the second cooling zone 79.

The second valve 87, third valve 84, fourth valve 90 and eighth valve 94 are closed.

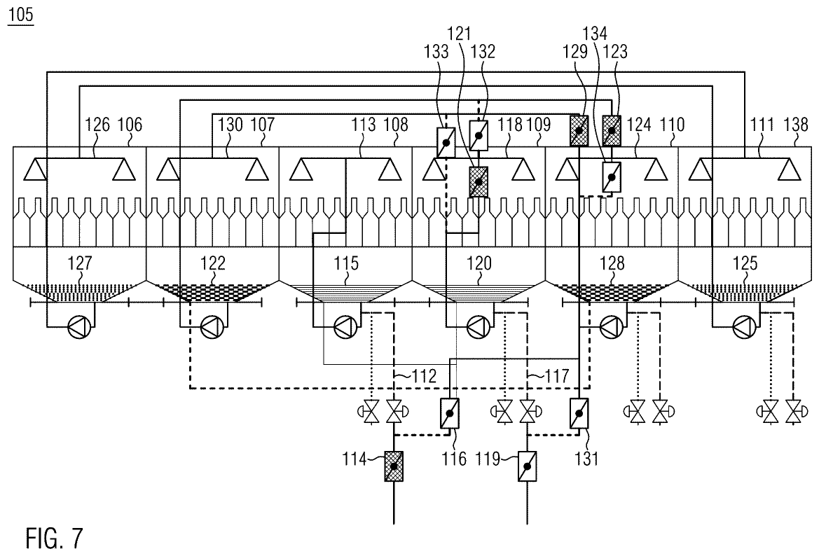

FIG. 7 shows a switching arrangement 105 for a displacement of a recuperation zone at 100% transport velocity in a pasteurizer, which comprises two heating zones 106, 107, two pasteurization zones 108, 109, and two cooling zones 110, 111. An operation with 100% transport velocity can be regarded as normal operation.

The supply line 112 to the spray device 113 of the first pasteurization zone 108 may be supplied with heated treatment medium via an open first valve 114, which medium is mixed with treatment medium 115 from the first pasteurization zone 108 and may then be supplied to the spray device 113 of the first pasteurization zone 108. A third valve 116 is closed.

The supply line 117 to the spray device 118 of the second pasteurization zone 109 may be supplied with heated treatment medium via an open second valve 119, which medium can be mixed with treatment medium 120 from the second pasteurization zone 109, and may then be supplied to the spray device 118 of the second pasteurization zone 109 via an open fifth valve 121.

From the first cooling zone 110, treatment medium 128 is supplied to the spray device 130 of the second heating zone 107 via an open ninth valve 129.

From the second heating zone 107, treatment medium 122 is supplied to the spray device 124 of the first cooling zone 110 via an open eighth valve 123.

From the second cooling zone 111, treatment medium 125 is supplied to the spray device 126 of the first heating zone 106.

From the first heating zone 106, treatment medium 127 is supplied to the spray device 138 of the second heating zone 111.

A fourth valve 131, a sixth valve 132, a seventh valve 133, and a tenth valve 134 are closed.

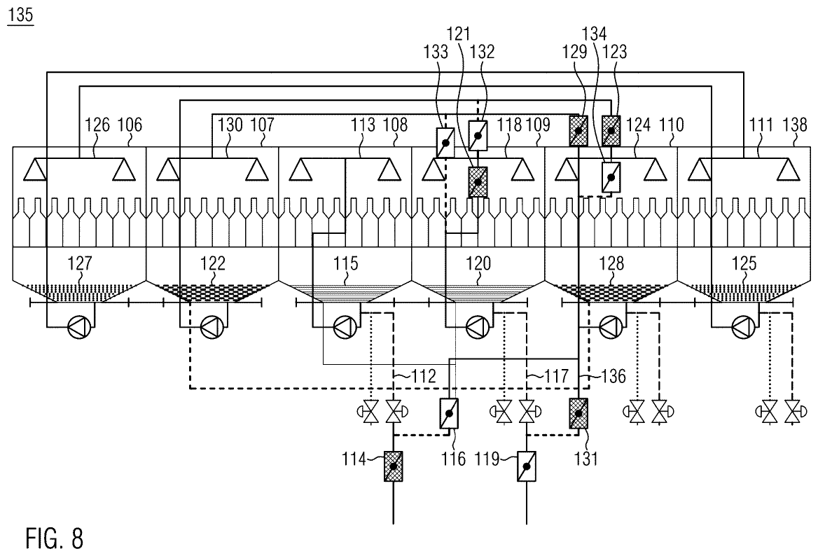

FIG. 8 shows a switching arrangement 135 for a displacement of the recuperation zone at 80% transport velocity in the pasteurizer which was described in FIG. 7.

The supply line 112 to the spray device 113 of the first pasteurization zone 108 may be supplied with heated treatment medium via the open first valve 114, which medium is mixed with treatment medium 115 from the first pasteurization zone 108 and may then be supplied to the spray device 113 of the first pasteurization zone 108. The third valve 116 is closed.

From the second pasteurization zone 109, treatment medium 120 is supplied to the spray device 118 of the second pasteurization zone 109 via the open fifth valve 121.

Heated treatment medium may be supplied to a discharge line 136 of the first cooling zone 110 via the open fourth valve 131, which medium can be mixed with treatment medium 128 from the first cooling zone 110, and may then be supplied to the spray device 130 of the second heating zone 107 via the open ninth valve 129.

From the second heating zone 107, treatment medium 122 is supplied to the spray device 124 of the first cooling zone 110 via the opened eighth valve 123.

From the second cooling zone 111, treatment medium 125 is supplied to the spray device 126 of the first heating zone 106.

From the first heating zone 106, treatment medium 127 is supplied to the spray device 138 of the second heating zone 111.

The second valve 119, the sixth valve 132, the seventh valve 133, and the tenth valve 134 are closed.

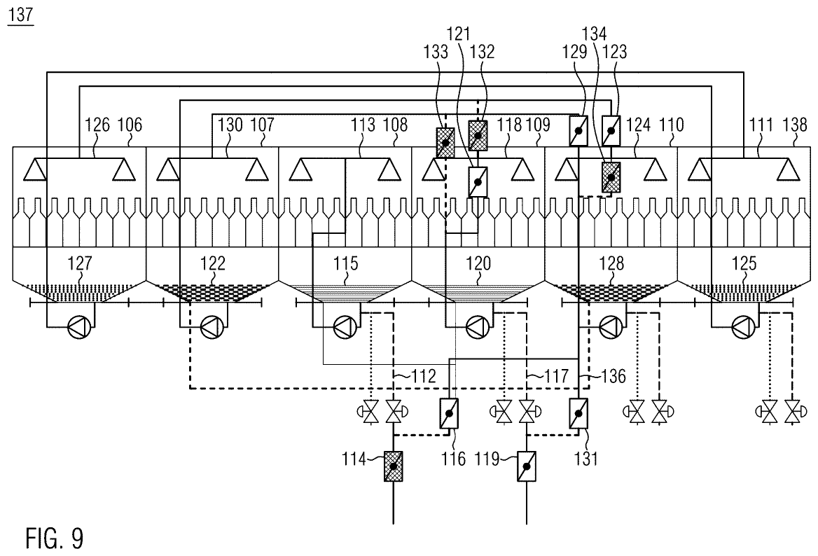

FIG. 9 shows a switching arrangement for a displacement of the recuperation zone at 50% transport velocity in the pasteurizer which was described in FIG. 7.

The supply line 112 to the spray device 113 of the first pasteurization zone 108 may be supplied with heated treatment medium via the open first valve 114, which medium is mixed with treatment medium 115 from the first pasteurization zone 108 and may then be supplied to the spray device 113 of the first pasteurization zone 108. The third valve 116 is closed.

From the second pasteurization zone 109, treatment medium 120 is supplied to the spray device 130 of the second heating zone 107 via the opened seventh valve 133.

From the second heating zone 107, treatment medium 122 is supplied to the spray device 118 of the second pasteurization zone 109 via the open sixth valve 132.

From the first cooling zone 110, treatment medium 128 is supplied to the spray device 124 of the first cooling zone 110 via the opened tenth valve 134.

From the second cooling zone 111, treatment medium 125 is supplied to the spray device 126 of the first heating zone 106.

From the first heating zone 106, treatment medium 127 is supplied to the spray device 138 of the second heating zone 111.

The second valve 119, the eighth valve 123 and the ninth valve 129 are closed.

1. A method for regulating a transport velocity in a pasteurizer, wherein the method comprises:
regulating the transport velocity by changing the transport velocity, making a corresponding first change of a first number of pasteurization zones of the pasteurizer by a second number, and making a corresponding second change of a third number of cooling zones of the pasteurizer by the second number.

2. The method according to claim 1, further comprising:
maintaining a fourth number of heating zones of the pasteurizer.

3. The method according to claim 1, wherein changing the transport velocity comprises reducing the transport velocity, wherein making the corresponding first change comprises reducing the first number of pasteurization zones of the pasteurizer by the second number, and wherein making the corresponding second change comprises increasing the third number of cooling zones of the pasteurizer by the second number.

4. The method according to claim 3, wherein reducing the first number of pasteurization zones of the pasteurizer by the second number, and increasing the third number of cooling zones of the pasteurizer by the second number comprises:
continuously lowering a first temperature in a last pasteurization zone, whereby the last pasteurization zone becomes a first cooling zone.

5. The method according to claim 4, wherein continuously lowering of the first temperature in the last pasteurization zone comprises:
converting a supply of a spray device in the last pasteurization zone with treatment medium from the last pasteurization zone into a supply of the spray device with treatment medium from the first cooling zone, and
converting a supply of a spray device in the last heating zone with treatment medium from the first cooling zone into a supply of the spray device with treatment medium from the last pasteurization zone.

6. The method according to claim 5, further comprising:
interrupting a separate supply of heated water into the last pasteurization zone.

7. The method according to claim 3, wherein reducing the first number of pasteurization zones of the pasteurizer by the second number, and increasing the third number of cooling zones of the pasteurizer by the second number is carried out by means of regulating multiple regulatable valves.

8. The method according to claim 1, wherein changing the transport velocity comprises increasing the transport velocity, wherein making the corresponding first change comprises increasing the first number of pasteurization zones of the pasteurizer by the second number, and wherein making the corresponding second change comprises reducing the third number of cooling zones of the pasteurizer by the second number.

9. The method according to claim 8, wherein increasing the first number of pasteurization zones of the pasteurizer by the second number and decreasing the third number of cooling zones of the pasteurizer by the second number comprises:
continuously increasing a first temperature in a first cooling zone, whereby the first cooling zone becomes a last pasteurization zone.

10. The method according to claim 1, wherein the transport velocity is changed continuously.

11. A device for carrying out the method according to claim 1, wherein the device comprises:
a pasteurizer having the first number of pasteurization zones and the third number of cooling zones,
a transport device which is designed to transport containers at the transport velocity in a transport direction through the pasteurizer,
a control device configured for changing the transport velocity, making the corresponding first change of the first number of pasteurization zones of the pasteurizer by the second number, and making the corresponding second change of the third number of cooling zones of the pasteurizer by the second number.

12. The device according to claim 11, wherein the pasteurizer comprises a fourth number of heating zones.

13. The device according to claim 11, wherein the device comprises multiple regulatable valves.

14. The device according to claim 13, wherein the multiple regulatable valves are arranged in pressure piping system of spray devices.

15. The device according to claim 11, further comprising a conversion device for converting a supply of a spray device in a last pasteurization zone with treatment medium from the last pasteurization zone into a supply of the spray device with treatment medium from a first cooling zone, and further comprising a second conversion device for converting a supply of a second spray device in the last heating zone with treatment medium from the first cooling zone into a supply of the spray device with treatment medium from the last pasteurization zone.

16. The device according to claim 11, wherein the device further comprises a heat exchanger for heating treatment medium.

17. The device according to claim 11, wherein the device further comprises a further heat exchanger for cooling treatment medium.

18. The method according to claim 10, wherein the transport velocity is changed infinitely variably, or wherein the transport velocity is changed in 0.5%, 1% or 1.5% increments.

19. The device according to claim 14, wherein the multiple regulatable valves comprise flap valves.
